(19) United States
(12) Patent Application Publication
(10) Pub. No.: US 2007/0160589 A1
(43) Pub. Date: Jul. 12, 2007

(54) Probiotic food, process for its preparation and dietary regimen

(75) Inventor: Peter H. Mattson, Hillsborough, CA (US)

Correspondence Address: MORRISON & FOERSTER LLP, 425 MARKET STREET, SAN FRANCISCO, CA, 94105-2482, US

(73) Assignee: ATTUNE FOODS, Mill Valley, CA (US)

(21) Appl. No.: 11/653,334

(22) Filed: Jan. 11, 2007

Related U.S. Application Data
(60) Provisional application No. 60/743,117, filed on Jan. 11, 2006.
(60) Provisional application No. 60/858,736, filed on Nov. 13, 2006.

(51) Int. Cl. A61K 35/74; A61K 47/00

(52) U.S. Cl. 424/93.45; 424/439

(57) ABSTRACT

A probiotic food item containing a beneficial amount of dry active probiotic cultures is provided. The food item also contains a substantial continuous fat-based coating with an effectively low water activity level. The food item is packaged in a substantially moisture impermeable package marked with a use by or sell by date, so as to ensure a desired minimal amount of probiotic colony forming units (CFUs) on the use by or sell by date. Methods for manufacturing the probiotic food product are also provided.

### CROSS REFERENCE TO RELATED APPLICATIONS

This application claims the benefit under 35 USC 119(e) of U.S. Provisional Application No. U.S. Patent Application No. 60/743,117, filed Jan. 11, 2006, and U.S. Provisional Application No. 60/858,736 was filed on Nov. 13, 2006 all of which are incorporated herein by reference in their entirety.

### FIELD OF THE INVENTION

The following disclosure is directed to a probiotic food item containing active, beneficial microbial cultures in stable form.

### BACKGROUND OF THE INVENTION

Probiotics are bacterial cultures that support a good and healthy intestinal bacterial flora. The term “probiotic” literally means “for life.” They include live, microbial cultures consumed or applied for a health benefit. Most probiotic products contain the bacteria from the genera Lactobacillus or Bifidobacterium, although other genera, including Escherichia, Enterococcus, and Saccharomyces (a yeast) have been identified as probiotics. They have also been reported to enhance the digestion and absorption of protein, fat, calcium and phosphorus, and they produce their own lactase and may help overcome lactose intolerance.

Current probiotic product offerings include yogurts, kefirs, cultured dairy drinks, and capsules. Some of these have amounts of probiotics that may not be significant and others may contain excessive dosages. When taking the probiotics as a supplement, it is important in the beginning to be cautious and take a low dosage. An overdose may trigger an excessive drainage syndrome, causing side effects such as headache, diarrhea or constipation.

Probiotic bacteria have a long history of association with fermented dairy products. Yogurt and kefir are milk based and fermented. They are believed to have originated with the discovery that fresh milk carried in leather pouches would occasionally ferment and provide stability. The kefir was the result of the milk fermenting to form an effervescent beverage. The process for preparing kefir now involves fermenting milk with what are called kefir grains, which are a mass of bacteria, yeast and polysaccharide. The grains have been found to contain Saccharomyces delbruecki, Saccharomyces cerevisiae, Lactobacillus kefir and members of the Streptococcus genera. The polysaccharide component has been called kefiran. The fermentation process takes about twenty-four hours, during which milk is converted into a thick, astringent tasting drink. Yogurt is a familiar fermented pudding-like product, which is prepared by fermenting milk with culture derived from an earlier batch. Kefir and yogurt are both probiotics because they contain live cultures. Being live, very high in moisture and in need of refrigeration, the cultures do not last long in yogurt and kefir.

Of high importance is the ability to provide some assurance to the consumer that the product in the store and when properly stored at home will have the desired activity in terms of the number of viable organisms. Food products, which are generally preferred to capsule and other oral supplement formats, do not generally indicate levels of bacteria on the label. Some supplements claim levels of 0.1-10 billion viable organisms while dairy products with probiotics are believed to contain about 200-300 million per cup. One milk-based product, Dannon's DanActive® cultured dairy drink (Actimel® in Europe), is said to contain 10 billion bacteria per serving. One study conducted by consumerlabs.com concluded that one-third of probiotic products do not contain the levels of active bacteria they claim. It would be desirable to have a product with a longer shelf life than dairy-based probiotic foods and one that is reliably stable over long periods of time.

The art has endeavored to provide stability, but a combination of a stable probiotic component and a pleasant, convenient dosage form is required to assure maintenance of a regimen where that would be beneficial. However, there remains a need for advances.

The art is in need of a probiotic food product which is pleasant to eat but that also, when provided in a convenient form, maintains a desirable level of active cultures over a substantial amount of time.

### BRIEF DESCRIPTION OF THE INVENTION

In order to meet the demand for a probiotic food product in a convenient form, the present disclosure provides a single serving, discrete probiotic food item that is substantially solid at room temperature. In one aspect, the food item contains a dry active probiotic culture dispersed in a fully enveloping, substantially continuous fat-based coating; the coating has a water activity between 0.1 to about 0.5; the food item is at a temperature of between about 0° F. and about 50° F. and is packaged in a moisture impermeable package that includes a use by or sell by date; and the food item contains at least 0.4 billion CFUs of culture per gram on the use by or sell by date.

In a variation of the probiotic food item, the item contains at least 0.6 billion CFUs of culture per gram on the use by or sell by date. In another variation the food item contains at least 0.8 billion CFUs of culture per gram on the use by or sell by date. In yet another variation, the food item contains at least 1 billion CFUs of culture per gram on the use by date or sell by date.

In one embodiment, an enrobing, fat-based coating of the probiotic food item has a water activity from about 0.2 to about 0.4. In a variation, the probiotic food item is at a temperature between about 33 and about 50° F. In another variation, the probiotic food item is at a temperature between about 40 and about 45° F. In one embodiment, the probiotic food item is in the form of a bar from about 10 to about 45 grams in weight. In another variation, the food item is a bar from about 10 to about 25 grams in weight. In yet another variation, the probiotic food item contains chocolate or a chocolate substitute. In another variation, the probiotic food item contains a calcium supplement. In yet another variation, the food item contains a prebiotic such as lactose, tagatose, or dietary fiber. In one variation, the prebiotic is insulin, a dietary fiber. In a variation, the probiotic food item contains a compressed matrix of one or more of: whole grains, nuts, rice crisps, puffed wheat, millet, soy, and dried fruit in the form of a bar enrobed in the fat-based coating. In yet another variation, the culture of the probiotic food item contains Lactobacillus acidophilus, Bifidobacterium lactis or Lactobacillus casei microorganisms.

### DETAILED DESCRIPTION OF THE INVENTION

The present disclosure enables the above objectives and provides a variety of benefits by providing a conveniently packaged, probiotic food containing a significant amount of stabilized live cultures and methods of manufacturing the same. This packaged food product facilitates maintenance of a regimen for intake of probiotics which may be beneficial and provides the consumer with a strong feeling of confidence that the product is providing the probiotic benefit desired.

Probiotic Culture

Essential to the food products disclosed herein is a suitable probiotic culture. The cultures will contain at least one non-pathogenic microorganism effective as a probiotic in humans. Preferably, the microorganisms will include bacteria including but not limited to Lactobacilli, Bifidobacteria, Streptococci, Pediococci, Leuconostoc, propionic and acetic bacteria. Among the Lactobacilli include but are not limited to Lactobacillus acidophilus, Lactobacillus bifidus, Lactobacillus brevis, Lactobacillus bulgaricus, Lactobacillus delbrucki, Lactobacillus casei, Lactobacillus cellobiosus, Lactobacillus fermentum, Lactobacillus gasseri, Lactobacillus germentum, Lactobacillus helveticus, Lactobacillus johnsonhii, Lactobacillus lactis, Lactobacillus leichimanii, Lactobacillus plantarum, Lactobacillus reuteri, Lactobacillus rhamnosus, Lactobacillus sake, Lactobacillus salivaroes, Lactobacillus thermophilus and Lactobacillus xylosus. Among the Bifidobacteria are those including but not limited to Bifidobacterium adolescentis, Bifidobacterium bifidum, Bifidobacterium breve, Bifidobacterium cereus, Bifidobacterium infantis, Bifidobacterium lactis, Bifidobacterium longum, and Bifidobacterium thermophilus. Among the Streptococci bacteria are those including but not limited to Streptococcus lactis, Streptococcus cremoris, Streptococcus diacetylactis, Streptococcus thermophilus, and Streptococcus faecium. Preferably, one or more cultures of any of these strains with another from this group or another probiotic, non-pathogenic microorganism may be employed. One preferred form includes a combination of probiotic species available from DSM Food Specialties®, The Netherlands, such as Lactobacillus acidophilus L10, Bifidobacterium lactis. B94, and Lactobacillus casei L26. In one embodiment, the microorganisms used include the Lactobacillus acidophilus strain and/or the Lactobacillus heveticus strain disclosed in International Application Publication WO 2006/048446 A1, naming DSM IP Assets B.V. as applicant which is hereby incorporated by reference in its entirety. Preferably, the probiotic culture will be in dry, viable form.

Storage temperatures for the cultures are preferably from about 39° to about 46° F., but can vary with the culture selected. The cultures are desirably prepared by freeze drying and can be effectively stored as recommended by the manufacturer. Among the commercial sources are DSM®, Dansico®, Institut Rossell-Lallemand, and others.

The packaged, probiotic culture items or products described herein will preferably contain at least about 2 billion colony forming units (CFU) per serving, at the end of shelf life. Colony-forming unit (CFU) is a measure of viable bacterial numbers. Unlike in direct microscopic counts where all cells, dead and living, are counted, CFU measures viable cells. In one format, a sample is spread or poured on a surface of an agar plate, left to incubate and the number of colonies formed are counted. CFU is not an exact measure of numbers of viable cells, as a colony-forming unit may contain any number of cells. A “serving,” as used herein, refers to a single portion of food or a helping as customarily taken at a particular time. The upper level of activity can be 20 billion CFU or higher per serving, with the main consideration here being the most suitable dosage form and the effect on flavor. A “discrete serving” is a serving apart or detached from others. Typically, the probiotic products of this disclosure will contain about 0.4 billion CFU per gram to about 1 billion CFU per gram, at the end of shelf life. Accordingly, preferred products will contain about 10 billion CFU per serving of from about 10 to about 25 grams, e.g., about 20 grams.

It is an advantage of preferred forms of the probiotic product described that they will contain more than about 1 to about 20 times, preferably more than about 2 to about 10 times, the live active cultures typically found in yogurt at the time of consumption. Such amounts find use in, among other things, helping to restore weakened immune systems. These ratios are based on the standard that fresh yogurt will contain at least 100 million organisms per gram at the time of manufacture for a 170 gram serving, but that activity typically goes down by a factor of 10 (e.g., from 100 million to 10 million live active cultures per gram) or so at the time of consumption so that guarantees at consumption are deemed impractical. (See e.g., a letter from the National Yogurt Association to the Food and Drug Administration, dated Jan. 27, 2004, captioned Comments to the Advanced Notice of Proposed Rulemaking for Milk and Cream Products and Yogurt Products; Petition to Revoke Standards for Lowfat Yogurt and Nonfat Yogurt and to Amend Standards for Yogurt and Cultured Milk (Docket No. 2000P-0685) (available on the world wide web at the FDA web site).

Fat-Based Coating and Flavoring

It is an advantage of the present disclosure that enrobing or encapsulating the probiotic cultures in a fat-based coating enables long-term stability. By “enrobing” or “encapsulating” it is meant that the probiotic culture is dispersed within and fully enveloped by the fat-based coating. By “enveloped” it is meant to enclose or enfold completely within the fat-based coating. The fat-based coating also enables both the use of these yogurt-based microorganisms in the presence of a chocolate or compound coating without the need for acid for stability or flavor and the use of acid flavors without changing the nature of the cultures. Chocolate substitutes, which are chocolate-like and mimic the appearance and flavor of chocolate, may also be employed. Thus, the formulations can be changed in production from either sweet to sour or vice versa without changing the stability of the cultures. The product can be made delicious with long term stability, as the low water activity (Aw) fat-based coating protects the dry cultures from moisture. It is an added advantage that manufacturing costs may be reduced because, due to the high stability of the product, there is a greatly reduced need to provide an excess of culture during manufacture to assure a desired activity at the time of consumption.

The probiotic culture product of this disclosure will include a continuous portion of a fat-based coating including a coating fat and, preferably, a sweetener. An edible emulsifier, as well as other ingredients, may also be present in the fat-based coating.

Any suitable fat-based coating, such as chocolate and chocolate substitutes, can be employed. Typically, and preferably, the coatings employed will be substantially solid at room temperature, where they will break with a snap, and melt in the mouth at just under body temperature. “Room temperature,” as used herein, refers to indoor temperatures commonly encountered in modern food markets, typically on the order of about 75° F. Those skilled in the art are aware of the fats and other components of such coatings. A typical coating fat will have a melting point of about 90° to about 98° F. and a solids content of at least about 50% at about 50° F. The term “fat-based coating” includes chocolate and so-called compound coatings, among others, and is typically characterized by a substantially continuous fat phase, having dispersed therein a sweetener, such as a sugar and/or intensive sweetener, and flavor components such as butterscotch and ground cocoa typically supplied as cocoa liquor as well as other flavors. The fat-based coatings can include a fat-based matrix and can be employed to incorporate both fat and water-soluble flavors. Preferably, the coatings will include sugar in the form of sucrose in a fat matrix comprised of cocoa butter or cocoa butter substitute, and the desired flavor components.

A typical chocolate-like confectionery composition suitable for use in preparing the products of the disclosure will contain fat, a sweetener, flavors and emulsifiers. For a typical chocolate flavored fat based coating, from about 5% to about 40% by weight will include chocolate flavoring (including chocolate liquor or cocoa which contain some inherent fat), about 25% to about 45%, e.g., from about 30 to about 35%, by weight fat ingredients, and about 0.001% to 40% by weight sweetener and/or bulking agent.

The fat component can be cocoa butter, palm kernel oil specially prepared for use as a coating fat, or any other fat or fat blend effective for forming a coating. The fat can be partially or wholly in the form of low-calorie fat or fat substitute such as olestra or salatrim. The preferred model for coating fats in terms of the solid fat index (SFI) is cocoa butter—that is, a steep curve with a melt temperature below 98° F. Cocoa butter can and usually is replaced and simulated in properties by other fats. Soybean and cottonseed oils can be prepared with suitable properties, as can palm oil, palm kernel oil, coconut oil, shea nut butter, illipe oil, among others.

Among the sweeteners are nutritive carbohydrate sweeteners, which are available with varying degrees of sweetness intensity. The probiotic food product of the disclosure is not restricted to any particular type of sweetener other than the requirement that it permits the formation of a low-moisture, low water activity (Aw) coating. Among those useful in the food product described herein are those typically used in the coating art and include, but are not limited to, sucrose, dextrose, fructose, lactose, maltose, glucose, glucose syrup solids, corn syrup solids, invert sugar, hydrolyzed lactose, honey, maple sugar, brown sugar, molasses and the like. The high intensity sweeteners include (where approved) aspartame, cyclamates, saccharin, acesulfame-K, sucralose, neohesperidin dihydrochalcone, alitame, stevia compositions, glycyrrhizin, thaumatin, and the like and mixtures thereof. A sugar alcohol such as mannitol or sorbitol can be substituted for a nutritive carbohydrate sweetener such as sucrose. If a high intensity sweetener is desired, a low-calorie bulking agent such as polydextrose can be employed in amounts similar to those used for sugar in more typical formulations. A sugar substitute or sweet sugar alcohol may partially replace all or part of the nutritive carbohydrate sweetener. Examples of sugar alcohols are any of those typically used in the art and include sorbitol, mannitol, xylitol, maltitol, isomalt, lactitol, and the like. Bulking agents as defined herein may be any of those typically used in the art and include polydextrose, cellulose, and its derivatives, maltodextrin, gum arabic and the like. The preferred sweetener is sucrose.

Another ingredient preferably present in the coatings utilized in the present probiotic food product is an edible emulsifier. Emulsifiers which may find use in the probiotic foods of the present disclosure may be any of those typically used in the art and include, but are not limited to, lecithin derived from vegetable sources, such as soybean, safflower, corn, etc.; fractionated lecithins enriched in either phosphatidyl choline or phosphatidyl ethanolamine, or both; mono and diglycerides thereof; monosodium phosphate derivatives of mono and diglycerides of edible fats or oils; lactylated fatty acid esters of glycerol and propylene glycol; hydroxylated lecithins; polyglycerol esters of fatty acids; propylene glycol; mono and diester of fats and fatty acids; DATEM (diacetyl tartaric acid esters of mono and diglycerides); PGPR (polyglycerol polyricinoleate); polysorbate 60, 65 and 80; sorbitan monostearate; sorbitan tristearate, oat extract; and the like.

The fat-based coatings described herein typically have a low Aw, or water activity, so as to minimize degradation during storage. The term “water activity” and the notation “Aw” as used herein refer to and are defined to be equal to the Equilibrium Relative Humidity (“ERH”) divided by 100. ERH is the equilibrium state at which the product neither absorbs nor loses moisture to the environment. The ERH is influenced by the composition of all ingredients, particularly those with high water contents, which may be present as free or bound water. The amount of free water influences the storage capabilities of the product which could result in undesired degradation of activity during storage.

Typically, the fat-based coatings used herein will have a water activity (Aw) of less than about 0.5 and most preferably less than about 0.1. Preferably, the probiotic food product having a fat-based coating with a low Aw may be stored under the recommended conditions for 1 to about 12 months or more.

It is preferred that the probiotic culture products disclosed herein remain essentially dry, and that they contain no more than a trace of water. The use of substantial quantities of water in processing is typically incompatible with the coating fats and the product stability.

Other optional ingredients, normally found in food product coatings may additionally be present. These optional ingredients include, but are not limited to, non-fat milk solids, non-fat cocoa solids, sugar substituents, natural and artificial flavors, such as vanillin, spices, coffee, ethyl vanillin, salt, brown nut-meats, natural vanilla and the like or combinations thereof, antioxidants, (e.g. preservatives, such as TBHQ (t-butyl hydroquinone), tocopherols, and the like), proteins, and the like. Among coating flavors are cocoa, chocolate flavor, dry coffee powder, freeze dried (FD) peach powder, peach flavor, vanilla flavor OS, annatto extract, citric acid, FD strawberry powder, strawberry flavor, and the like. In alternate embodiments, suitable flavor compositions are effective flavoring amounts of at least one member selected from the group consisting of buttery flavors, vanilla flavors, cream dairy flavors, caramel-like flavors, and other flavors associated with freshness. To some extent these flavors and their perceptions overlap, but the test for whether one is present in amounts sufficient for any of these flavor effects will be best based upon suitable instrumentally derived data (e.g., gas chromatography) and expert flavor panel evaluation. Specifically identified as buttery flavors are diacetyl, acetoin, acetol, butyric acid, gamma undecalactone, gamma nonalactone, delta decalactone, acetyl methyl carbinol, lipolized butter fat and mixtures of two or more of these. Representative of vanilla flavors are vanilla bean extract, vanillin, ethyl vanillin, maltol, dihydro coumarin, heliotropin and mixtures of two or more of these. Exemplary of creamy or cream dairy flavoring are vanillin, ethyl vanillin, maple lactone, diacetyl, butyric acid, gamma undecalactone, gamma nonalactone, delta decalactone, acetyl methyl carbinol, caproic acid, lipolized butter fat and mixtures of two or more of these. Examples of caramel flavorings include vanillin, ethyl vanillin, gamma undecalactone, dihydro coumarin, caramel color and mixtures of two or more of these. Other desirable flavors can comprise any other freshness notes, such as dimethylsulfide, ethyl butyrate, benzaldehyde, butyl butyryl lactate, oil of nutmeg, lemon oil and the like.

In one embodiment, the method of encapsulating or enrobing in a fat-based substance is that described in International Application Publication WO 2006/018119 A1, naming DSM IP Assets B.V. as applicant which is hereby incorporated by reference in its entirety.

Inclusions

The probiotic food product described herein may also contain inclusions, preferably present as dry pieces of suitable foods to be incorporated with the fat-based coating to add textural and/or flavor interest. Suitable inclusion materials will “interrupt” the homogeneous composition of the coating portion when introduced into the formulation. These materials are essentially inert, so far as the coating or probiotic are concerned. Examples of such materials, referred to hereinafter as “inclusions”, are expanded cereals, e.g., puffed oats, wheat or rice, crisped rice or the like; extruder expanded dough pieces, e.g., of wheat, soy, other grain or the like; cereal flakes, e.g., of corn, wheat, rice, bran, oats or the like; oatmeal or rolled oats; chopped nuts or pieces of nuts such as pecans, hazelnuts, walnuts, peanuts or other types of nuts; raisins or other dried fruits or fruit pieces such as of apple, banana, blueberry, cranberry, currant, date, mango, orange, pineapple, raspberry, strawberry or sultana; fruit flavored morsels; coconut; hard or soft candy pieces; other small pieces of contrasting flavor or texture; and the like.

Dietary Supplements

Preferably, the probiotic food product of the disclosure will contain significant dietary amounts of calcium, in addition to the probiotic culture. Preferably, they will contain at least about 10% of the DV (“daily value”) of calcium, e.g., at least about 20% (200 mg) of the DV for calcium. The term “daily value” (“DV”) is given the meaning employed by the U.S. FDA and explained in a circular by Paula Kurtzweil entitled “Daily Values Encourage Healthy Diet”: (www.fda.gov/fdac/special/foodlabel/dvs.html). The circular explains that DRVs (daily recommended values) serve as the basis for calculating percent DV. DRVs are stated to be for nutrients for which no set of standards previously existed. DRVs for the energy-producing nutrients (fat, carbohydrate, protein, and fiber) are based on the number of calories consumed per day. For labeling purposes, 2,000 calories has been established as the reference for calculating percent DVs. Thus, those consumers used to taking a calcium supplement as part of their daily routine, will find it easy to supplant that with the calcium-containing, probiotic food product of the present disclosure which not only supplies the calcium, but tastes extremely good and provides an excellent, stable source of probiotics. Any of the typical sources of dietary calcium may be employed, but it is preferred to use those from dairy sources, such as calcium lactate, calcium caseinate, and the like. In addition to these sources, the more typical calcium carbonate, e.g., from shells, and the like may also be employed. In alternative embodiments, other dietary supplements known to those skilled in the art may be included.

Prebiotics

The probiotic food products disclosed herein may contain quantities of prebiotics. Prebiotics are substances, typically not digested in the stomach of the host, which promote the growth of certain intestinal bacteria. Prebiotics which may be included in the food products described are sugars, such as lactose or tagatose, and dietary fiber. Other prebiotics known in the art may also be included.

The probiotic food products of the present disclosure may be advantageously formulated with dietary fiber as a prebiotic and can accomplish this without significant decrease in stability, as will normally occur when fiber is blended with probiotic cultures in other products. The fiber, thus, contributes to the healthfulness of the product without diminishing the stability of the probiotics. The result is a more beneficial product that helps to promote a healthy intestinal bacterial flora. The fiber can be provided from any suitable source, including that derived from various vegetable and fruit sources, including purified vegetable fiber, such as insulin extracted from the roots of the chicory plant; various fruit pulps and vegetable pulps available largely as byproducts of juicing or other food preparation operations, such as apple pulp, citrus pulp, mango pulp, grape pulp, cranberry pulp and beet pulp; and fruit and vegetable purees, such as those prepared from fresh and/or frozen fruits and vegetables.

Manufacture

The probiotic foods described herein are delicious to encourage their consumption, which can provide a snack or meal introduction having a significant concentration of viable organisms and preferably other nutrients, in a convenient and stable form. They can be prepared simply, but the process is quite important to the product stability. In one preferred form, the process entails melting the coating fat at a suitable temperature, but not too high to cause processing delays or inefficiencies. For example, palm kernel oil can be fully melted at about 110 to about 130° F., e.g., about 120° F. To this melted fat, can be added nutrients such as fiber and calcium supplements, as well as color, flavors and the like. The mixture of fat and added ingredients is preferably blended until homogenous. Following blending, the resulting blend is partially cooled to a temperature fully compatible with the probiotic cultures, which are mixed sufficiently to assure complete coating with the fat. Following this operation, the inclusions, if any, are added and folded in. The final mixture is then ready for molding, which is desired for portion control.

Standard manufacturing procedures known in the art of food science, such as procedures for the manufacture of confectionary bars and snack bars, may be employed. Particularly, the procedures in Bernard Minifie, CHOCOLATE, COCOA, AND CONFECTIONERY: SCIENCE AND TECHNOLOGY (Springer 3rd ed., 1989)(1970) which is hereby incorporated by reference in its entirety.

Packaging and Use

A preferred format for provision of the probiotic food item of the present disclosure is a single serving bar of about 20 grams, which is preferably packaged in a moisture impermeable package such as a substantially moisture proof film, e.g., of metallized multi-layer polymer film. Preferably, the film will have a moisture or water vapor transfer rate of 0.0 grams/(meter²×day). Standard film materials known in the art that provide a good moisture barrier, such as a flow wrap which is cold sealed, may be used.

The probiotic food product described herein, when packaged and stored as described, will have, at the end of shelf life, about 0.4 billion CFU per gram, more preferably 0.6 billion CFU per gram, even more preferably 0.8 CFU per gram, and most preferably about 1 billion CFU per gram.

To ensure the desired CFU content at the end of shelf life, the probiotic food product is preferably formulated at time of manufacture with about 0.5 billion CFU per gram, more preferably 0.8 billion CFU per gram, even more preferably 1.0 CFU per gram, and most preferably about 1.2 billion CFU per gram. Doing so allows for a degree of culture loss during storage.

The packaging and the water activity of the food are preferably selected to assure stability of the culture at refrigerated temperature for at least one month, preferably at least 3 months, preferably at least 9 months, and even more preferably at least 12 months, with less than about 20%, or even more preferably 10%, loss in viable cultures, as measured in CFUs. By way of comparison, fresh yogurt typically has a refrigerated shelf life of 45 days. “Refrigerated temperature” or “refrigerated storage,” as used herein, refers to storage at temperatures typical of modern grocery store refrigeration, e.g. from about 0° F. to about 50° F., more preferably about 33° F. to about 50° F.

In a preferred embodiment, a “use by” or “sell by” date is marked on the individual packages. A “use by” date typically denotes a date pertinent to the end consumer, by which the product should be consumed. The “use by” date typically indicates the end of shelf life. A “sell by” date typically denotes a date pertinent to the end retailer, by which the product should be sold; typically, a product may be consumed several days after the “sell by” date. It is understand that other language which conveys the same information as a “use by” or “sell by date,” such as “best by,” “enjoy by,” “best enjoyed by” or “expires on” dates may also be used. In the context of the probiotic food items described herein, the “use by” or “sell by” date designates the period in which a desired minimal level of viable cultures (CFUs) is ensured.

It is preferred to utilize the probiotic food products of the disclosure in a regimen of regular consumption, e.g., at least twice weekly, preferably at least 5 times per week, and more preferably on a daily basis so as to promote a healthy intestinal bacterial flora.

### EXAMPLES

The following examples are provided to further illustrate and explain the probiotic products described herein and their methods of manufacture, without being limiting in any regard. Unless otherwise indicated, all parts and percentages are based on the weight of the composition at the particular point of reference.

### Example 1

A chocolate-flavored confectionary bar containing a mixed culture with 26 billion CFU per bar was prepared from the following formulation and procedure.

TABLE 1
Chocolate-flavored bar

| Ingredient | Percent |
|---|---|
| Chocolate Coating, see table below | 94.382% |
| Crisp Rice | 4.743% |
| Lactobacillus acidophilus L10 (DSM) | 0.800% |
| Bifidobacterium sp. B94 (DSM) | 0.042% |
| Lactobacillus casei L26 (DSM) | 0.033% |
| | 100.000% |

TABLE 2
Chocolate coating

| Ingredient | Percent |
|---|---|
| Sugar | 38.40% |
| Chocolate Liquor | 24.51% |
| Cocoa Butter | 17.65% |
| Inulin | 8.82% |
| Non-Fat Yogurt Powder | 4.90% |
| Calcium Carbonate | 2.94% |
| Anhydrous Butter oil | 2.45% |
| Soy Lecithin | 0.32% |
| Ground Vanilla Bean | 0.29% |
| | 100.000% |

Procedure:
1. Melt coating to 120° F.
2. Cool to between 95° F. and 110° F. and add probiotic cultures.
3. Temper chocolate coating.
4. Fold in rice crisps.
5. Deposit into molds and cool at 65° F.
6. Unmold and package.

### Example 2

A blueberry-flavored confectionary bar containing a mixed culture with 26 billion CFU per bar was prepared from the following formulation and procedures.

TABLE 3
blueberry-flavored bar

| Ingredient | Percent |
|---|---|
| White Coating, see table below | 93.046% |
| Crisp Rice | 4.396% |
| Freeze-Dried Blueberries | 1.122% |
| Blueberry Flavor | 0.561% |
| Lactobacillus acidophilus L10 (DSM) | 0.800% |
| Bifidobacterium sp. B94 (DSM) | 0.042% |
| Lactobacillus casei L26 (DSM) | 0.033% |
| | 100.000% |

TABLE 4
White coating

| Ingredient | Percent |
|---|---|
| Sugar | 35.00% |
| Cocoa Butter | 29.00% |
| Non-Fat Yogurt Powder | 14.00% |
| Whole Milk Powder | 9.50% |
| Inulin | 9.00% |
| Calcium Carbonate | 3.00% |
| Soy Lecithin | 0.30% |
| Ground Vanilla Bean | 0.20% |
| | 100.000% |

Procedure:
1. Melt coating to 120° F.
2. Cool to between 95° F. and 110° F. and add blueberry flavor and probiotic cultures.
3. Temper chocolate coating.
4. Fold in rice crisps and freeze-dried blueberries.
5. Deposit into molds and cool at 65° F.
6. Unmold and package.

### Example 3

A strawberry-flavored granola bar topped with a confectionary coating containing a mixed culture with 14 billion CFU per bar was prepared from the following formulation and procedures.

TABLE 5
Strawberry-flavored granola bar

| Ingredient | Percent |
|---|---|
| Granola Bar, see table below | 80.00% |
| White Coating with Probiotics, see below | 20.00% |
| | 100.000% |

TABLE 6
Granola Bar

| Ingredient | Percent |
|---|---|
| Granola, see table below | 53.16% |
| Syrup, see table below | 46.84% |
| | 100.000% |

TABLE 7
Granola

| Ingredient | Percent |
|---|---|
| Honey Granola | 42.04% |
| Almonds | 18.02% |
| Soy Crisps | 12.01% |
| Crisp Brown Rice | 10.51% |
| Dried Cranberry Halves | 9.01% |
| Sunflower Seeds | 6.01% |
| Freeze-Dried Strawberries | 2.40% |
| | 100.000% |

TABLE 8
Syrup

| Ingredient | Percent |
|---|---|
| Brown Rice Syrup | 51.40% |
| Yogurt Powder | 8.52% |
| Inulin | 8.52% |
| Palm Shortening | 6.82% |
| Whey Protein Isolate | 6.82% |
| Glycerin | 6.82% |
| Evaporated Cane Juice | 5.11% |
| Calcium Carbonate | 2.39% |
| Strawberry Flavor | 1.64% |
| Citric Acid, 50% Solution | 1.60% |
| Salt | 0.27% |
| Soy Lecithin | 0.10% |
| | 100.000% |

TABLE 9
White Coating with Probiotics

| Ingredient | Percent |
|---|---|
| Compound Yogurt Coating | 98.813% |
| Lactobacillus acidophilus L10 (DSM) | 1.000% |
| Bifidobacterium sp. B94 (DSM) | 0.104% |
| Lactobacillus casei L26 (DSM) | 0.083% |
| | 100.000% |

Procedure:
Syrup:
1. Blend liquid syrup ingredients and evaporated cane juice and heat to 120° F.
2. Add yogurt powder, whey protein, insulin, and calcium carbonate to syrup and mix into a smooth slurry
   White Coating with Probiotics:
1. Melt compound yogurt coating to between 95° F. and 110° F.
2. Add probiotic cultures and blend thoroughly.
   Granola Bar:
1. Blend all granola ingredients.
2. Add heated syrup to granola ingredients and mix until evenly coated.
3. Press granola and syrup mixture to form a slab.
4. After set, cut into 32 gram bars and bottom coat with 8 grams of white coating with probiotics.
5. Package.

The above description is for the purpose of teaching the person of ordinary skill in the art how to utilize the disclosure provided herein. It is not intended to detail all of those obvious modifications and variations which will become apparent to the skilled worker upon reading the description. It is intended, however, that all such obvious modifications and variations be included within the scope the following claims. The claims are meant to cover the claimed components and steps in any sequence which is effective to meet the objectives there intended, unless the context specifically indicates the contrary.

1. A discrete probiotic food item comprising:
a dry probiotic culture dispersed within and fully enveloped by a substantially continuous fat-based coating;
wherein water activity of the fat-based coating is from about 0.1 to about 0.5; and
wherein the probiotic food item is at a temperature of between about 0° F. and about 50° F. and is packaged in a substantially moisture impermeable package;
wherein the substantially moisture impermeable package includes a use by or sell by date;
wherein the culture comprises live cultures in an amount of at least about 0.4 billion colony forming units (CFUs) per gram of the food item on the use by or sell by date; and
wherein the probiotic food item is substantially solid at room temperature.

2. A food item according to claim 1, wherein the culture comprises live cultures in an amount of at least about 0.6 billion CFUs per gram of the food item on the use by or sell by date.

3. A food item according to claim 1, wherein the culture comprises live cultures in an amount of at least about 0.8 billion CFUs per gram of the food item on the use by or sell by date.

4. A food item according to claim 1, wherein the culture comprises live cultures in an amount of at least about 1 billion CFUs per gram of the food item on the use by or sell by date.

5. A food item according to claim 1, wherein the water activity of the coating is from about 0.2 to about 0.4.

6. A food item according to claim 1 wherein the temperature is between about 33° F. and about 50° F.

7. A food item according to claim 1 wherein the temperature is between about 40° F. and about 45° F.

8. A food item according to claim 1 wherein the food item is in the form of a bar of from about 10 to about 45 grams in weight.

9. A food item according to claim 1 wherein the food item is in the form of a bar of from about 10 to about 25 grams in weight.

10. A food item according to claim 1, wherein the fat-based coating comprises chocolate or chocolate substitute.

11. A food item according to claim 1, further comprising a calcium supplement.

12. A food item according to claim 1, further comprising a prebiotic.

13. A food item according to claim 11, wherein the prebiotic is selected from the group consisting of: lactose, tagatose, and dietary fiber.

14. A food item according to claim 13, wherein the prebiotic is a dietary fiber.

15. A food item according to claim 14, wherein the dietary fiber is insulin.

16. A food item according to claim 1, further comprising an inclusion selected from the group consisting of: a compressed matrix of whole grains, nuts, rice crisps, puffed wheat, millet, soy, and dried fruit.

17. A food item according to claim 1, wherein the dry active culture comprises a microorganism selected from the group consisting of: Lactobacillus acidophilus, Bifidobacterium lactis, and Lactobacillus casei.
